(19) United States
(12) Patent Application Publication
(10) Pub. No.: US 2012/0085358 A1
(43) Pub. Date: Apr. 12, 2012

(54) Systems and Methods for Magnetically Charging and Discharging a Member Configured for Medical Use

(76) Inventors: Jeffery Cadeddu, Dallas, TX (US); Daniel J. Scott, Dallas, TX (US); Raul Fernandez, Arlington, TX (US); Heather Beardsley, Arlington, TX (US); Richard Bergs, Grand Prarie, TX (US)

(21) Appl. No.: 12/899,327

(22) Filed: Oct. 6, 2010

(51) Int. Cl. A61B 17/00; H01F 13/00

(52) U.S. Cl. 128/899; 335/284

(57) ABSTRACT

Systems and methods for magnetically charging and discharging a member are disclosed. In certain embodiments, an external apparatus or internal device may comprise the member.

### BACKGROUND OF THE INVENTION

1. Field of the Invention

The present invention relates generally to medical devices, apparatuses, systems, and methods, and, more particularly, but not by way of limitation, to medical devices, apparatuses, systems, and methods for performing medical procedures at least partially within a body cavity of a patient.

2. Description of Related Art

For illustration, but without limiting the scope of the invention, the background is described with respect to medical procedures (e.g., surgical procedurals), which can include laparoscopy, transmural surgery, and endoluminal surgery, including, for example, natural orifice transluminal endoscopic surgery (NOTES), single-incision laparosopic surgery (SILS), and single-port laparoscopy (SLP).

Compared with open surgery, laparoscopy can result in significantly less pain, faster convalescence and less morbidity. NOTES, which can be an even less-invasive surgical approach, may achieve similar results. Recently, surgical techniques have been developed that use a magnet external to the body cavity to manipulate a surgical device within the body cavity. The surgical device may be introduced to the body cavity via a natural orifice or laparoscopically.

The apparatus used to manipulate the surgical device may comprise one or more magnets. In some instances, these magnets are rare-earth magnets with a strong magnetic field. Unintended attractions may result between the apparatus and ferromagnetic objects in the surgical environment.

### SUMMARY OF THE INVENTION

Any embodiment of any of the present methods, systems, and devices can consist of or consist essentially of—rather than comprise/include/contain/have—any of the described elements and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Details associated with the embodiments described above and others are presented below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers.

FIG. 1 depicts one embodiment of a system comprising a surgical device positioned within the body cavity of a patient and a positioning apparatus located outside the body cavity magnetically coupled to the surgical device.

FIGS. 2A and 2B depict a side view and a top view, respectively, of one embodiment of a cylindrical magnet.

FIGS. 3A and 3B depict one embodiment of an electrically conductive coil and a substantially-uniform magnetic field generated by the coil.

FIG. 4 depicts one embodiment of a magnetizer and a fixture.

FIGS. 5A and 5B depict one embodiment of a cylindrical magnet in the substantially-uniform magnetic field at a first orientation.

FIGS. 6A and 6B depict one embodiment of a cylindrical magnet in the substantially-uniform magnetic field at a second orientation.

FIG. 7 depicts an orientation that may be used to charge one embodiment of a cylindrical magnet and an orientation that may be used to discharge the cylindrical magnet.

FIGS. 8A-8D depict one embodiment of a prismatic magnet and orientations that may be used to charge and discharge it.

FIGS. 9A-9C depict one embodiment of a magnet having a specialized shape and example orientations that may be used to charge and discharge it.

FIGS. 10A-10C depict embodiments of orientations that may be used to charge and discharge a magnet having a specialized shape.

### DESCRIPTION OF ILLUSTRATIVE EMBODIMENTS

The term “coupled” is defined as connected, although not necessarily directly, and not necessarily mechanically; two items that are “coupled” may be integral with each other. The terms “a” and “an” are defined as one or more unless this disclosure explicitly requires otherwise. The terms “substantially,” “approximately,” and “about” are defined as being largely but not necessarily wholly what is specified, as understood by a person of ordinary skill in the art.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system or device that “comprises,” “has,” “includes” or “contains” one or more elements possesses those one or more elements, but is not limited to possessing only those one or more elements. Likewise, a method that “comprises,” “has,” “includes” or “contains” one or more steps possesses those one or more steps, but is not limited to possessing only those one or more steps. Similarly, an element of a system, device, or method that “comprises,” “has,” “includes” or “contains” one or more features possesses those one or more features, but is not limited to possessing only those one or more features. For example, a system that comprises an apparatus that includes a member and a fixture configured to generate a substantially-uniform magnetic field space that can induce a magnetic charge in the member includes the member and the fixture but is not limited to only having the member and fixture. They system could also include, for example, a magnetizer.

Further, a device or structure that is configured in a certain way is configured in at least that way, but it can also be configured in other ways than those specifically described.

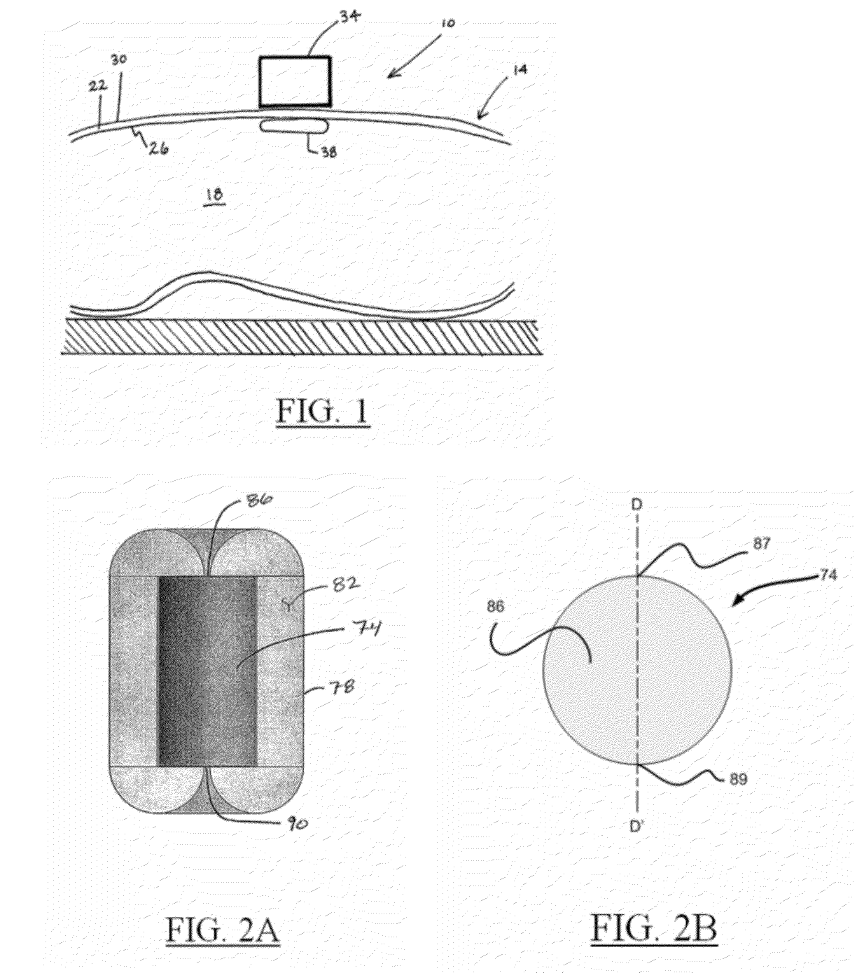

Referring now to the drawings, shown in FIG. 1 by reference numeral 10 is one embodiment of a system for medical procedures that can be used with the present invention. FIG. 1 shows system 10 in operation. System 10 is shown in conjunction with a patient 14, and more particularly in FIG. 1 is shown relative to a longitudinal cross-sectional view of the ventral cavity 18 of a human patient 14. For brevity, cavity 18 is shown in simplified conceptual form without organs and the like. Cavity 18 is at least partially defined by wall 22, such as the abdominal wall, that includes an interior surface 26 and an exterior surface 30. The exterior surface 30 of wall 22 can also be an exterior surface 30 of the patient 14. Although patient 14 is shown as human in FIG. 1, various embodiments of the present invention (including the version of system 10 shown in FIG. 1) can also be used with other animals, such as in veterinary medical procedures.

Further, although system 10 is depicted relative to ventral cavity 18, system 10 and various other embodiments of the present invention can be utilized in other body cavities of a patient, human or animal, such as, for example, the thoracic cavity, the abdominopelvic cavity, the abdominal cavity, the pelvic cavity, and other cavities (e.g., lumens of organs such as the stomach, colon, or bladder of a patient). In some embodiments of the present methods, and when using embodiments of the present devices and systems, a pneumoperitoneum may be created in the cavity of interest to yield a relatively-open space within the cavity.

As shown in FIG. 1, system 10 comprises an apparatus 34 and a medical device 38. Apparatus 34 is configured to magnetically position device 38 with a body cavity of a patient. In some embodiments, apparatus 34 can be described as an exterior apparatus and/or external unit and device 38 as an interior device and/or internal unit due the locations of their intended uses relative to patients. Apparatus 34 and device 38 are both examples of structures configured for use in a medical or surgical procedure. As shown, apparatus 34 can be positioned outside the cavity 18 near, adjacent to, and/or in contact with the exterior surface 30 of the patent 14. Device 38 is positionable (can be positioned), and is shown positioned, within the cavity 18 of the patient 14 and near, adjacent to, and/or in contact with the interior surface 26 of wall 22. Device 38 can be inserted or introduced into the cavity 18 in any suitable fashion. For example, the device 18 can be inserted into the cavity through a puncture (not shown) in wall 22, through a tube or trocar (not shown) extending into the cavity 18 through a puncture or natural orifice (not shown), or may be inserted into another portion of the patient 14 and moved into the cavity 18 with apparatus 34, such as by the methods described in this disclosure. If the cavity 18 is pressurized, device 38 can be inserted or introduced into the cavity 18 before or after the cavity 18 is pressurized. Additionally, some embodiments of system 10 include a version of device 38 that has a tether (not shown) coupled to and extending away from the device 38.

In the embodiment shown, apparatus 34 and device 38 comprise one or more members that are configured to be magnetically charged, magnetically discharged, or both. These members are referred to generally as “magnets,” even though at various times their magnetic charge may be substantially zero. The magnets may comprise, for example, Ferrite, such as can comprise Barium or Strontium; AlNiCo, such as can comprise Aluminum, Nickel, and Cobalt; SmCo, such as can comprise Samarium and Cobalt and may be referred to as rare-earth magnets; and NdFeB, such as can comprise Neodymium, Iron, and Boron. In some embodiments, it can be desirable to use magnets of a specified grade, for example, grade 40, grade 50, or the like. Such suitable magnets are currently available from a number of suppliers, for example, Magnet Sales & Manufacturing Inc., 11248 Playa Court, Culver City, Calif. 90230 USA; Amazing Magnets, 3943 Irvine Blvd. #92, Irvine, Calif. 92602; and K & J Magnetics Inc., 2110 Ashton Dr. Suite 1A, Jamison, Pa. 18929. In some embodiments, one or more magnetic field sources can comprise ferrous materials (e.g., steel) and/or paramagnetic materials (e.g., aluminum, manganese, platinum).

As is discussed in more detail below, apparatus 34 and device 38 can be configured to be magnetically couplable to one another such that device 38 can be positioned or moved within the cavity 18 by positioning or moving apparatus 34 outside the cavity 18. “Magnetically couplable” means capable of magnetically interacting so as to achieve a physical result without a direct physical connection. Examples of physical results are causing device 38 to move within the cavity 18 by moving apparatus 34 outside the cavity 18, and causing device 38 to remain in a position within the cavity 18 or in contact with the interior surface 26 of wall 22 by holding apparatus 34 in a corresponding position outside the cavity 18 or in contact with the exterior surface 30 of wall 22. Magnetic coupling can be achieved by configuring apparatus 34 and device 38 to cause a sufficient magnetic attractive force between them. For example, apparatus 34 can comprise one or more magnets and device 38 can comprise a ferromagnetic material. In some embodiments, apparatus 34 can comprise one or more magnets, and device 38 can comprise a ferromagnetic material, such that apparatus 34 attracts device 38 and device 38 is attracted to apparatus 34. In other embodiments, both apparatus 34 and device 38 can comprise one or more magnets such that apparatus 34 and device 38 attract each other.

The configuration of apparatus 34 and device 38 to cause a sufficient magnetic attractive force between them can be a configuration that results in a magnetic attractive force that is large or strong enough to compensate for a variety of other factors (such as the thickness of any tissue between them) or forces that may impede a desired physical result or desired function. For example, when apparatus 34 and device 38 are magnetically coupled as shown, with each contacting a respective surface 26 or 30 of wall 22, the magnetic force between them can compress wall 22 to some degree such that wall 22 exerts a spring or expansive force against apparatus 34 and device 38, and such that any movement of apparatus 34 and device 38 requires an adjacent portion of wall 22 to be similarly compressed. Apparatus 34 and device 38 can be configured to overcome such an impeding force to the movement of device 38 with apparatus 34. Another force that the magnetic attractive force between the two may have to overcome is any friction that exists between either and the surface, if any, that it contacts during a procedure (such as apparatus 34 contacting a patient's skin).

In some embodiments, device 38 can be inserted into cavity 18 through an access port having a suitable internal diameter. Such access ports includes those created using a conventional laparoscopic trocar, gel ports, those created by incision (e.g., abdominal incision), and natural orifices. Device 38 can be pushed through the access port with any elongated instrument such as, for example, a surgical instrument such as a laparoscopic grasper or a flexible endoscope.

In some embodiments, when device 38 is disposed within cavity 18, device 38 can be magnetically coupled to apparatus 34. This can serve several purposes including, for example, to permit a user to move device 38 within cavity 18 by moving apparatus 34 outside cavity 18. The magnetic coupling between the two can be affected by a number of factors, including the distance between them. For example, the magnetic attractive force between device 38 and apparatus 34 increases as the distance between them decreases. As a result, in some embodiments, the magnetic coupling can be facilitated by temporarily compressing the tissue (e.g., the abdominal wall) separating them. For example, after device 38 has been inserted into cavity 18, a user (such as a surgeon) can push down on apparatus 34 (and wall 22) and into cavity 18 until apparatus 34 and device 38 magnetically couple.

In FIG. 1 apparatus 34 and device 38 are shown at a coupling distance from one another and magnetically coupled to one another such that device 38 can be moved within the cavity 18 by moving apparatus 34 outside the outside wall 22. The “coupling distance” between two structures (e.g., apparatus 34 and device 38) is defined as a distance between the closest portions of the structures at which the magnetic attractive force between them is great enough to permit them to function as desired for a given application.

The “maximum coupling distance” between two structures (e.g., apparatus 34 and device 38) is defined as the greatest distance between the closest portions of the structures at which the magnetic attractive force between them is great enough to permit them to function as desired for a given application. Factors such as the thickness and composition of the matter (e.g., human tissue) separating them can affect the coupling distance and the maximum coupling distance for a given application. For example, in the embodiment shown in FIG. 1, the maximum coupling distance between apparatus 34 and device 38 is the maximum distance between them at which the magnetic attractive force is still strong enough to overcome the weight of device 38, the force caused by compression of wall 22, the frictional forces caused by contact with wall 22, and any other forces necessary to permit device 38 to be moved within cavity 18 by moving apparatus 34 outside wall 22. In some embodiments, apparatus 34 and device 38 can be configured to be magnetically couplable such that when within a certain coupling distance of one another the magnetic attractive force between them is strong enough to support the weight of device 38 in a fixed position and hold device 38 in contact with the interior surface 26 of wall 22, but not strong enough to permit device 38 to be moved within the cavity 18 by moving apparatus 34 outside wall 22.

In some embodiments, apparatus 34 and device 38 can be configured to have a minimum magnetic attractive force at a certain distance. For example, in some embodiments, apparatus 34 and device 38 can be configured such that at a distance of 50 millimeters between the closest portions of apparatus 34 and device 38, the magnetic attractive force between apparatus 34 and device 38 is at least about: 20 grams, 25 grams, 30 grams, 35 grams, 40 grams, or 45 grams. In some embodiments, apparatus 34 and device 38 can be configured such that at a distance of about 30 millimeters between the closest portions of apparatus 34 and device 38, the magnetic attractive force between them is at least about: 25 grams, 30 grams, 35 grams, 40 grams, 45 grams, 50 grams, 55 grams, 60 grams, 65 grams, 70 grams, 80 grams, 90 grams, 100 grams, 120 grams, 140 grams, 160 grams, 180 grams, or 200 grams. In some embodiments, apparatus 34 and device 38 can be configured such that at a distance of about 15 millimeters between the closest portions of apparatus 34 and device 38, the magnetic attractive force between them is at least about: 200 grams, 250 grams, 300 grams, 350 grams, 400 grams, 45 grams, 500 grams, 550 grams, 600 grams, 650 grams, 700 grams, 800 grams, 900 grams, or 1000 grams. In some embodiments, apparatus 34 and device 38 can be configured such that at a distance of about 10 millimeters between the closest portions of apparatus 34 and device 38, the magnetic attractive force between them is at least about: 2000 grams, 2200 grams, 2400 grams, 2600 grams, 2800 grams, 3000 grams, 3200 grams, 3400 grams, 3600 grams, 3800 grams, or 4000 grams. These distances may be coupling distances or maximum coupling distances for some embodiments.

Referring now to FIGS. 2A-2B, a side view and top view are shown of a magnet 74. Apparatus 34 or device 38 may comprise one or more magnets 74. In some embodiments, magnet 74 may be used in apparatus 34 to manipulate device 38 from outside the body. In some embodiments, apparatus 34 may comprise two magnets 74 where magnets 74 do not touch one another. In other embodiments, apparatus 34 may comprise a plurality of magnets 74 coupled end-to-end, such that the S end of one magnet 74 is coupled to the N end of another magnet 74. In other embodiments, magnet 74 may have a specialized shape. Magnet 74 may be configured to be housed within apparatus 34 and may not be removable or may not be readily removable. In other embodiments, magnet 74 may be configured to be removable from apparatus 34.

Magnet 74 may also be housed in, carried on, or physically coupled to device 38. Magnet 74 may be used in device 38 to magnetically couple device 38 to apparatus 34 such that device 38 may be manipulated in body cavity 18 by moving apparatus 34. In some embodiments, device 38 may comprise two magnets 74 that do not touch each other. In other embodiments, magnet 74 may be have a specialized shape. In other embodiments, device 38 may comprise a plurality of magnets 74 coupled end-to-end, such that the S end of one magnet 74 is coupled to the N end of another magnet 74. Magnet 74 may be configured to be housed within device 38 and may not be removable or may not be readily removable. In other embodiments, magnet 74 may be configured to be removable from device 38.

The embodiments illustrated in FIGS. 2A, 2B and 5A-7 depict cylindrical magnets, but magnets of all shapes may be used, including but not limited to prismatic, pyramidal, conical, rhomboid, and annular. Magnet 74 used in apparatus 34 may have a different shape than magnet 74 used in device 38. Also, more or fewer magnets may be used in apparatus 34 than may be used in device 38. For example, in some embodiments apparatus 34 may comprise two cylindrical magnets 74 that do not touch one another, while device 38 may comprise one specialized magnet 74. In other embodiments, apparatus 34 may comprise two prismatic magnets 74 that do not touch one another while device 38 may comprise two cylindrical magnets 74 that do not touch one another. In other embodiments, apparatus 34 may comprise a plurality of cylindrical magnets 74 stacked end-to-end such that an N-pole of one magnet touches an S pole of an adjacent magnet, and device 38 may comprise one cylindrical magnet 74.

As shown in FIG. 2A, magnet 74 has a first end 86 and a second end 90. Field lines 78 conceptually illustrate the magnetic field 82 of magnet 74. When a magnetic charge in induced in magnet 74 by exposing magnet 74 to a sufficiently strong magnetic field, magnet 74 will have an a N pole and an S pole. For a cylindrical magnet having a circular cross-sectional shape, such as magnet 74, the N and S poles may be aligned with the axis passing through the center of the circular cross-sectional shape. For example, when first end 86 is the N pole, second end 90 is generally the S pole; and where first end 86 is the S pole, second end 90 is generally the N pole. As conceptually illustrated by field lines 78, in the absence of external influences, magnetic field 82 is generally evenly distributed about magnet 74 and flows through the N and S poles. Although magnet 74 is shown as a single cylindrical cylinder, in some embodiments (not shown), magnet 74 can comprise a plurality of, for example, two, three, four, or more, shorter cylindrical magnets oriented in a linear configuration to form magnet 74. In such an embodiment, each shorter magnet can be magnetically charged to have an N and a S pole, and can be oriented such that the S pole of each shorter magnet is adjacent to the N pole of the next adjacent shorter magnet, such that each S pole attracts and is attracted to the next adjacent N pole.

As shown in FIG. 2B, in other embodiments, a magnet may be magnetized through its thickness. In the embodiments depicted, a cylindrical magnet may be magnetized along a diameter of its circular cross section. Line D-D′ passes through one diameter of magnet 74. First node 87 and second node 89 are the points along the surface of the cylinder at which a plane passing through one diameter of the circular cross-section of magnet 74 intersects the edges of magnet 74. The N and S poles may be aligned with the diameter in this embodiment. For example, when first node 87 is the N pole, second node 89 is generally the S pole. When first node 87 is the S pole, second node 89 is generally the N pole.

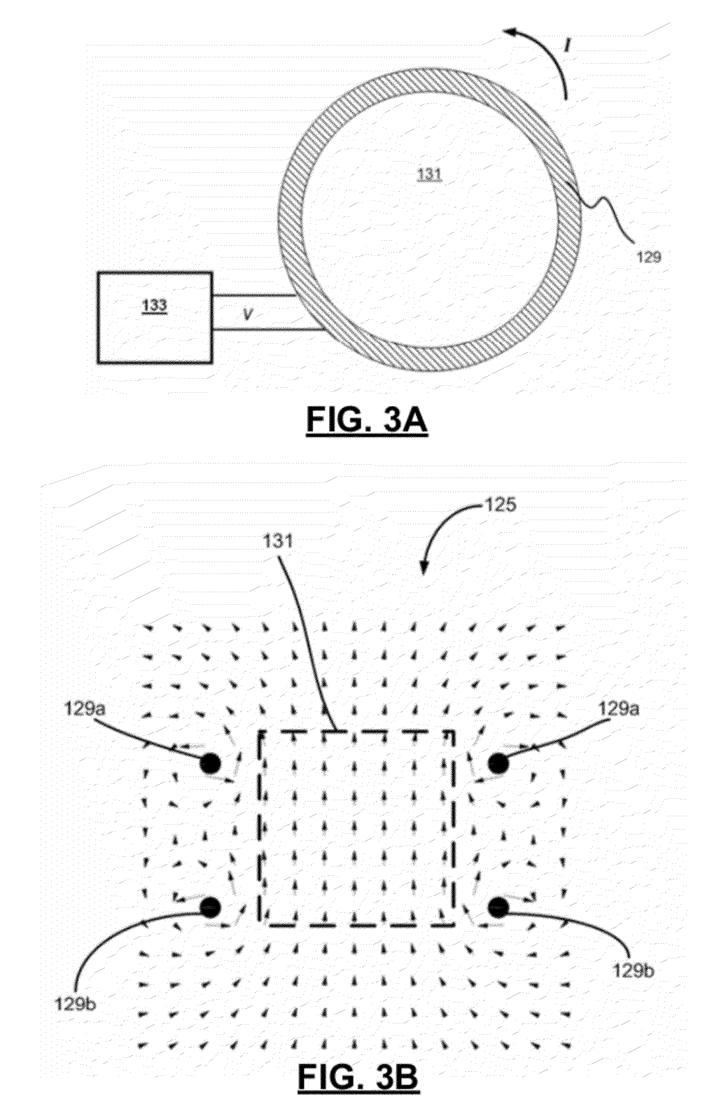

Referring now to FIGS. 3A and 3B, schematic views of a substantially uniform magnetic field generated by a plurality of electrically-conductive coils are shown. A magnet may be charged by exposing it to a sufficiently strong, substantially uniform magnetic field.

A magnetic material comprises a number of small volumes called domains. Each domain can be thought of as a tiny magnet with its own magnetic axis having an N pole and an S pole. In an uncharged state (also called an inert state or a demagnetized state), these axes point in many different directions, and are said to be unaligned.

Magnetic fields can be modeled as a vector field, with each vector having a magnitude and a direction. Magnetic fields display many of the same properties as vector fields, e.g. vectors may be added and subtracted, and vectors of equal magnitudes and opposing directions cancel. For example, a vector having a magnitude M and a direction (0, 0, z) added to a vector having a magnitude M and a direction (0, 0, −z) yields a vector with a magnitude of 0.

Like opposing vectors, the magnetic field of one magnetic domain cancels out the magnetic field of another magnetic domain aligned in the opposite direction. The net effect of these unaligned magnetic domains is that the magnetic material is substantially uncharged on a macro level. That is, the magnetic material has no discernable N pole or S pole. In some instances, the magnetic domains can be weakly aligned. As a result, the magnetic material has a weak magnetic field.

Introducing an uncharged magnetic material to a substantially uniform magnetic field will align the domains in the direction of the magnetic field, and a magnetic charge is induced in the material. Once a sufficient number of magnetic domains have aligned, the magnet is considered to be in a “charged” or “magnetized” state. Once all the magnetic domains have been aligned, the magnet is considered to be in a “saturated” state. The greater the number of magnetic domains that have been aligned, the stronger the magnetic field of the charged magnet.

Some magnetic domains align more easily than others. The type of material being magnetized, and the strength and uniformity of the magnetic field in which the material is placed, affect how many domains will align. Temperature is also a factor in aligning the domains. Generally, charging a magnet in a more uniform magnetic field will yield a magnet with a stronger charge. Thus, a strong, uniform magnetic field is usually preferable when charging a magnet.

Any method or means of generating a substantially uniform magnetic field may be used. One non-limiting example of a device capable of generating a substantially uniform magnetic field is a plurality of metal coils having an electric current. FIGS. 3A and 3B illustrate one embodiment of coils 129 configured to generate substantially-uniform magnetic field space 131.

FIG. 3A illustrates one coil 129. Coil 129 is substantially circular in the illustrated embodiments, but other configurations may be used, including square coils, rectangular coils, ovoid coils, C-coils, Bitter coils, Helmholtz coils, and Maxwell coils. Coil 129 comprises many windings of an electrical conductor. In some embodiments, the electrical conductor may comprise copper wire or insulated copper wire.

As shown in FIG. 3A, voltage source 133 having voltage Vis electrically coupled to coil 129 and is configured to generate electrical current I through coil 129. When electrical current I flows through coil 129, substantially-uniform magnetic field space 131 is generated in the space inside coil 129. Flowing electrical current I through coil 129 in a first direction (e.g., counterclockwise) generates a magnetic field space having a first orientation. Reversing the flow of electrical current I through coil 129 (e.g. clockwise) generates a magnetic field space having a second orientation diametrically opposed to the first orientation. The stronger the current I, the stronger the magnetic field in substantially-uniform magnetic field space 131. FIG. 3B illustrates substantially-uniform magnetic field space 131 as a plurality of vector arrows aligned in substantially the same direction. Cross-sectional top views of two coils 129a and 129b with current I flowing through them are shown. Outside of the coils, unaligned magnetic field 125 is illustrated with a plurality of vector arrows pointing in substantially different directions.

Voltage source 133 may be any device capable of delivering a voltage to an electrically conductive material. In some embodiments, voltage source 133 must be capable of delivering a high voltage V to generate a strong current I in coils 129.

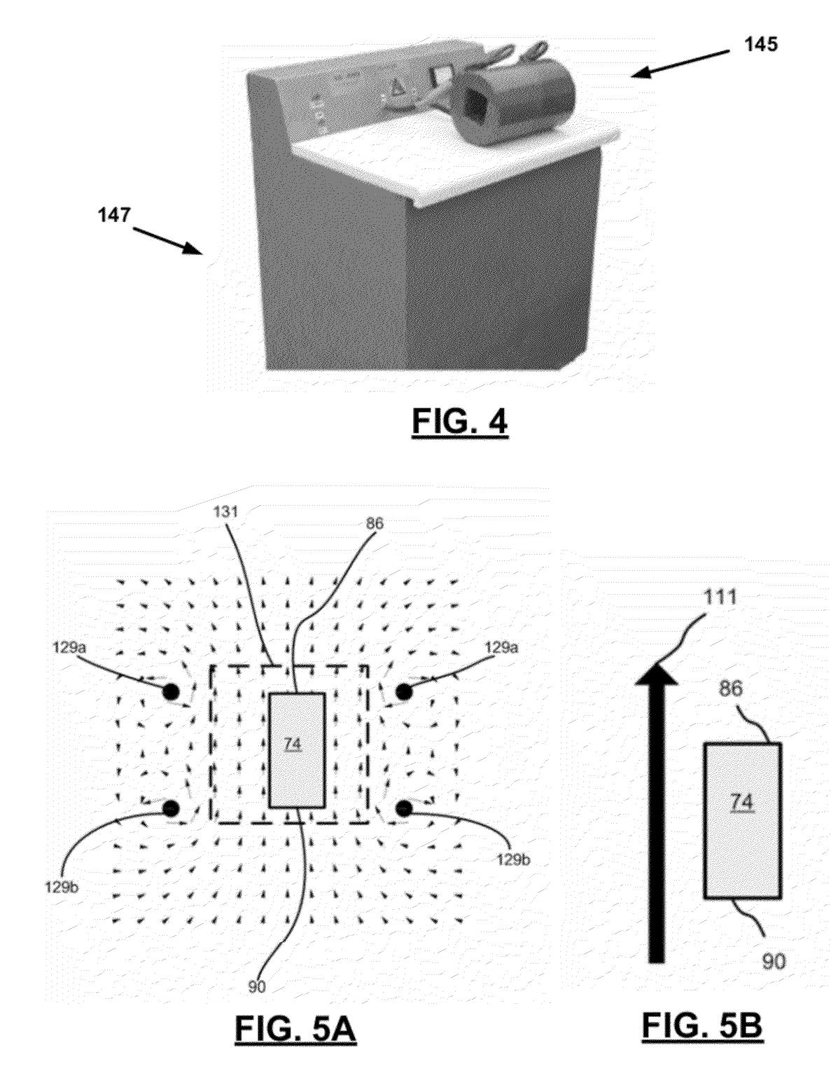

FIG. 4 illustrates one embodiment of magnetizer 147 and fixture 145. In some embodiments, voltage source 131 may be a magnetizer 147. In some embodiments, coil 129 may be housed in a fixture 145. In other embodiments, fixture 145 may be coil 129. Magnetizer 147 is electrically coupled to fixture 145 such that magnetizer 147 generates a voltage V and a current I in fixture 145.

In some embodiments, magnetizer 147 is configured to generate a direct current. In other embodiments, magnetizer 147 is configured to generate an alternating current. In some embodiments, magnetizer 147 is a capacitive-discharge magnetizer. Magnetizer 147 may operate in open-loop, such that voltage V is discharged according to a set rate and current curve to reach a desired saturation. In other embodiments, magnetizer 147 may operate in a closed-loop, such that the magnetizer measures the charge in the magnet and adjusts the strength of the substantially-uniform magnetic field space accordingly.

Magnetizer 147 may be configured to generate a current I in coil 129 of at least 5,000 A, 10,000 A, 15,000 A, 20,000 A, 25,000 A, 30,000 A, 35,000 A, 40,000 A, and 50,000 A. In some embodiments, a user may select a value for the current or the voltage delivered by magnetizer 147. In other embodiments, magnetizer 147 is configured to deliver a fixed voltage or current.

In some embodiments, fixture 145 may be configured to house one coil 129. In other embodiments, fixture 145 may be configured to house a plurality of coils 129. In other embodiments, fixture 145 may itself be coil 129. In some embodiments, fixture 145 is configured to generate substantially uniform magnetic field space 131. In some embodiments, fixture 145 is configured to receive magnet 74. Magnet 74 may be placed in a caddy configured to be inserted into fixture 145. In other embodiments, fixture 145 may be configured to receive apparatus 34 comprising magnet 74. In other embodiments, fixture 145 may be configured to receive device 38 comprising magnet 74. In some embodiments, apparatus 34 or device 38 may be placed in a caddy configured to be inserted into fixture 145.

Magnetizers and fixtures are readily available. Manufacturers of magnetizers and fixtures include: ALL Magnetics, Inc., 2831 Via Martens Anaheim, Calif. 92806; Master Magnetics, Inc., 607 S. Gilbert St. Castle Rock, Colo. 80104; Miami Magnet Co. 6073 NW 167th St., Ste. C26 Miami, Fla. 33015; and Oersted Technology, 24023 NE Shea Lane Unit #208, Troutdale, Oreg. 97060.

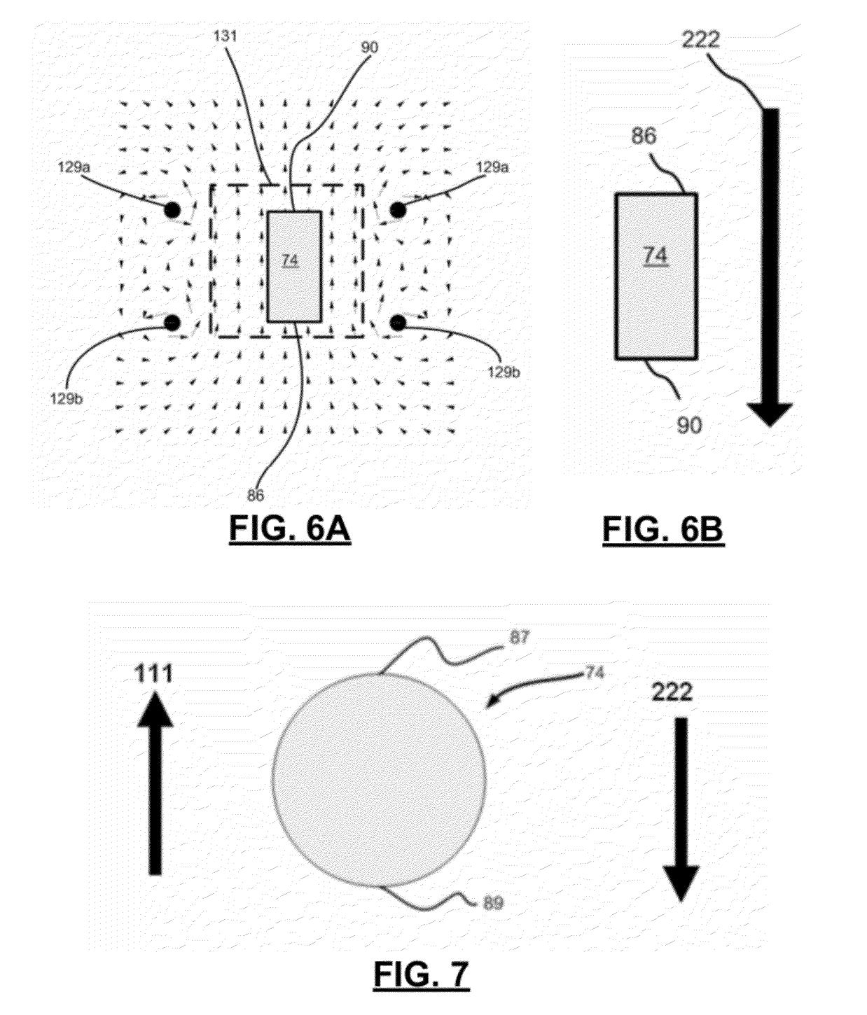

Referring now to FIGS. 5A-6B, schematic drawings illustrating charging and discharging magnet 74 are shown. In some embodiments, magnet 74 is configured to be removable from apparatus 34 or device 38, charged in substantially-uniform magnetic field space 131, and returned to apparatus 34 or device 38. In other embodiments, apparatus 34 comprising magnet 74 may be placed in substantially-uniform magnetic field space 131 with magnet 74 in or on apparatus 34. In other embodiments, device 38 comprising magnet 74 may be placed in substantially-uniform magnetic field space 131 with magnet 74 in or on device 38. Entire apparatus 34 or device 38 may be placed in substantially-uniform magnetic field space 131 to induce a charge in magnet 74.

FIGS. 5A and 5B illustrate magnet 74 in substantially-uniform magnetic field space 131 at a first orientation 111. For clarity, apparatus 34 or device 38 are not illustrated. However, apparatus 34 or device 38 may be present in substantially-uniform magnetic field space 131 with magnet 74. In first orientation 111, magnet 74 is aligned with substantially-uniform magnetic field space 131 along its length such that a vector originating at second end 90 and terminating at first end 86 is aligned with the direction of magnetic field vectors in substantially-uniform magnetic field space 131.

In some embodiments, magnet 74 is substantially discharged before it is introduced to substantially-uniform magnetic field space 131. A magnetic charge presents difficulties for shipping, storing, and cleaning magnet 74, or apparatus 34 comprising magnet 74, or device 38 comprising magnet 74. Unintended attractions between magnet 74 and metal objects can be hazardous. Thus, it may be desirable to charge magnet 74 on site, e.g., at the hospital or clinic where the surgery will be performed. Magnet 74 may be discharged on site for cleaning, sterilization, shipping, or storage. In other embodiments, magnet 74 may be initially substantially discharged, charged for use in surgery, and not discharged following surgery. In other embodiments, magnet 74 may be initially charged, used in surgery, then discharged following surgery.

Embodiments of the invention in which magnet 74 is initially discharged will be discussed. When magnet 74 is placed in substantially-uniform magnetic field space 131 at first orientation 111, magnet 74 becomes charged such that first end 86 and second end 90 become opposing magnetic poles. For example, in the embodiment shown in FIGS. 5A-5B, first end 86 may be an N pole and second end 90 may be an S pole. In other embodiments, first end 86 may be an S pole and second end 90 may be an N pole.

The strength of current I (see FIG. 3A) through coils 129a and 129b in the illustrated embodiment may be adjusted to induce a magnetic charge of a desired strength in magnet 74. In some embodiments, substantially-uniform magnetic field space 131 has a magnetic field strength of at least 1 kilooersted (kOe), 2 kOe, 3 kOe, 4 kOe, 5 kOe, 6 kOe, 7 kOe, 8 kOe, 9 kOe, 10 kOe, 11 kOe, 12 kOe, 13 kOe, 14 kOe, 15 kOe, 16 kOe, 17 kOe, 18 kOe, 19 kOe, 20 kOe, 21 kOe, 22 kOe, 23 kOe, 24 kOe, 25 kOe, 26 kOe, 27 kOe, 28 kOe, 29 kOe, 30 kOe, 31 kOe, 32 kOe, 33 kOe, 34 kOe, 35 kOe, 36 kOe, 37 kOe, 38 kOe, 39 kOe, or 40 kOe. The strength of the magnetic field used to charge magnet 74 is the “charge field strength.” The strength of the magnetic field used to discharge charged magnet 74 is the “discharge field strength.”

Once magnet 74 is charged, it may be used to, e.g., manipulate a surgical device in a patient using apparatus 34 comprising magnet 74. Or device 38 comprising magnet 74 may be inserted into a patient for surgery. In embodiments where magnet 74 is removable from apparatus 34 or device 38, magnet 74 may be inserted into apparatus 34 or device 38 before surgery is performed.

In some embodiments, magnet 74 may be discharged after use in, e.g., surgery. Referring now to FIGS. 6A and 6B, magnet 74 is shown in substantially-uniform magnetic field space 131 at second orientation 222. For ease of discussion, in this embodiment, first end 86 is an N pole and second end 90 is an S pole. In other embodiments, second end 90 could be an N pole and first end 86 could be an S pole. In embodiments where magnet 74 is substantially discharged, neither first end 86 nor second end 90 would be a magnetic pole.

In this embodiment, magnet 74 has a magnetic charge such that first end 86 is an N pole and second end 90 is an S pole. Charged magnet 74 may be placed in substantially-uniform magnetic field space 131 at second orientation 222. Second orientation 222 is diametrically opposed to first orientation 111. In other words, first orientation 111 and second orientation 222 are 180 degrees apart about a central axis of magnet 74. In embodiments where magnet 74 is charged along its length, the central axis is equidistant between first end 86 and second end 90 and normal to an axis along the length.

In the illustrated embodiment, magnetic field vectors in substantially-uniform magnetic field space 131 are oriented in S-to-N directions (that is, the tail of each arrow is S and the head of each arrow is N). Placing magnet 74 in substantially-uniform magnetic field space 131 in second orientation 222 orients magnet 74 in an N-to-S direction such that substantially-uniform magnetic field space 131 oriented in an S-to-N direction is an opposing magnetic field.

Placing charged magnet 74 in substantially-uniform magnetic field space 131 at second orientation 222 will discharge magnet 74 if the discharge field strength is approximately equal to the charge filed strength. Depending on the material of which magnet 74 is made, a weaker magnetic field may be used to discharge magnet 74 than was used to charge magnet 74. For example, the discharge field strength may be at least 95%, 90%, 85%, 80%, 75%, or 70% of the charge field strength. In some embodiments, magnet 74 is charged in a substantially-uniform magnetic field space 131 having a strength of 10 kOe. Magnet 74 might be discharged in a substantially-uniform magnetic field space 131 having a strength of at least 9.5 kOe, 9 kOe, 8.5 kOe, 8 kOe, 7.5 kOe, or 7 kOe.

Once magnet 74 is substantially discharged, apparatus 34 or device 38 may be sterilized, shipped, stored, or any or all of the preceding. In embodiments where magnet 74 is configured to be removable from apparatus 34 or device 38, magnet 74 may be sterilized, shipped, stored, or any or all of the preceding.

Exposing charged magnet 74 to an opposing magnetic field (that is, reversing the orientation of magnet 74 relative to the magnetic field) may be used to diminish the strength of a charged magnet 74 but not discharge it completely. In certain instances, magnet 74 may have a charge that is too strong for a desired application. Rather than discharging magnet 74 completely, it may be desirable to diminish the strength of its magnetic charge. In these instances, magnet 74 may be introduced to substantially-uniform magnetic field space 131 having a magnetic field strength substantially less than the magnetic field strength of the magnet. For example, magnet 74 may have a desired strength of 10 kOe, but has an actual strength of 12 kOe. Exposing magnet 74 to an opposing magnetic field having a strength of, e.g. 2 kOe will diminish the strength of the magnetic field of magnet 74, but the orientation of magnet 74 will not change and magnet 74 will not become completely discharged.

As shown in FIG. 7, magnet 74 may be charged through its thickness along a diameter, instead of through its length along a central axis. Magnet 74 may be aligned in first orientation 111 such that a vector originating at second node 89 and terminating at first node 87 would be aligned with the magnetic field vectors in substantially-uniform magnetic field space 131. Magnet 74 aligned to be charged through its thickness may be discharged by applying an opposing magnetic field of approximately equal strength as the magnetic field used to charge magnet 74 initially, as discussed above.

In the illustrated embodiments discussed so far, magnet 74 has been depicted as being cylindrical and charged along its length. In other embodiments, magnet 74 may have another shape and need not be charged along its length. For example, magnet 74 may be charged along its width or its thickness, or in any other suitable direction.

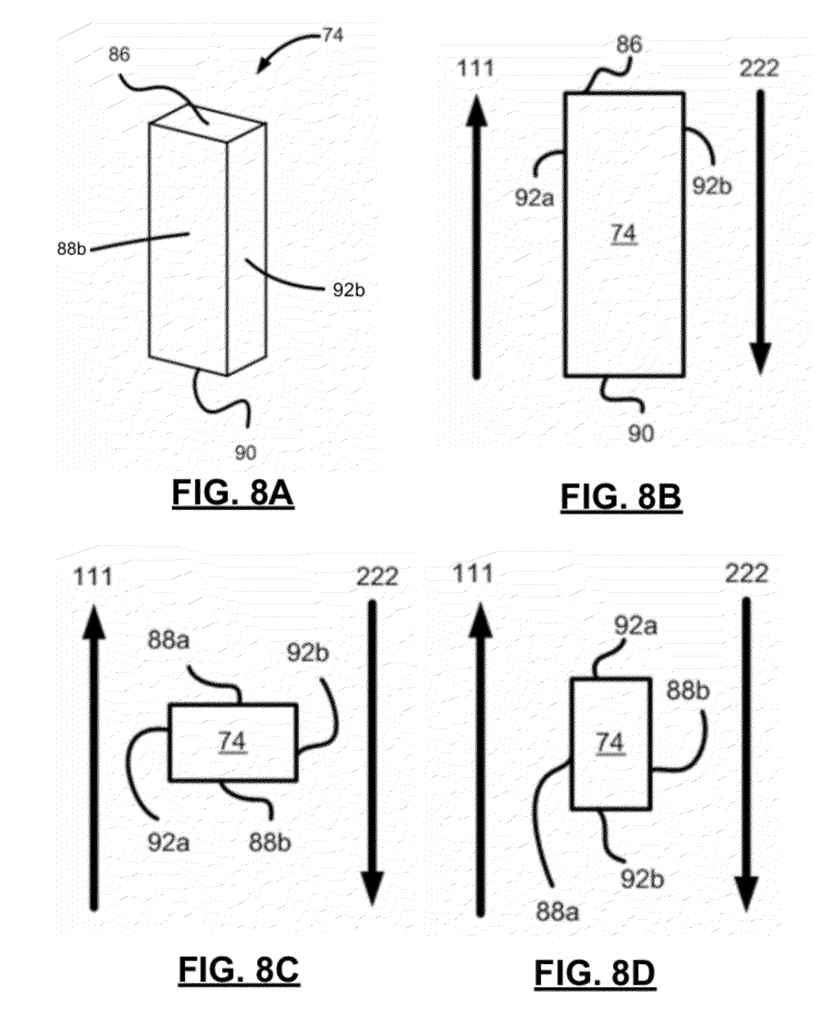

FIGS. 8A-10C illustrate several embodiments of magnet 74 configured to be charged in various orientations. Magnetic field lines, coils, and the like are not illustrated. One skilled in the art will appreciate that all of the embodiments of the members illustrated in FIGS. 8A-10C may be placed in a substantially-uniform magnetic field space as was discussed above with respect to cylindrical magnets.

FIGS. 8A-8D illustrate a perspective view, a side view, and two top views of a prismatic magnet 74. In this embodiment, magnet 74 comprises first end 86, second end 90, faces 88a and 8b and sides 92a and 92b. FIG. 8B shows magnet 74 configured (or positioned) to be charged and discharged along its length. In this configuration, in first orientation 111, magnet 74 is aligned with substantially-uniform magnetic field space 131 along its length such that a vector originating at second end 90 and terminating at first end 86 is aligned with the direction of magnetic field vectors in substantially-uniform magnetic field space 131. In some embodiments, first end 86 may be an N pole and second end 90 may be an S pole. In other embodiments, first end 86 may be an S pole and second end 90 may be an N pole. In second orientation 222, magnet 74 is aligned with substantially-uniform magnetic field space 131 along its length such that a vector originating at first end 86 and terminating at second end 90 opposes the direction of magnetic field vectors in substantially-uniform magnetic field space 131.

FIG. 8C shows magnet 74 configured to be charged and discharged through its thickness. In this configuration, in first orientation 111, magnet 74 is aligned with substantially-uniform magnetic field space 131 such that a vector originating at second face 88b and terminating at first face 88a is aligned with the direction of magnetic field vectors in substantially-uniform magnetic field space 131. In some embodiments, first face 88a may be an N pole and second face 88b may be an S pole. In other embodiments, first face 88a may be an S pole and second face 88b may be an N pole. In second orientation 222, magnet 74 is aligned with substantially-uniform magnetic field space 131 along its length such that a vector originating at first face 88a and terminating at second face 88b opposes the direction of magnetic field vectors in substantially-uniform magnetic field space 131.

FIG. 8D shows magnet 74 configured to be charged and discharged through its width. In this configuration, in first orientation 111, magnet 74 is aligned with substantially-uniform magnetic field space 131 such that a vector originating at second side 92b and terminating at first side 92a is aligned with the direction of magnetic field vectors in substantially-uniform magnetic field space 131. In some embodiments, first side 92a may be an N pole and second side 92b may be an S pole. In other embodiments, first side 92a may be an S pole and second side 92b may be an N pole. In second orientation 222, magnet 74 is aligned with substantially-uniform magnetic field space 131 along its length such that a vector originating at first side 92a and terminating at second side 92b opposes the direction of magnetic field vectors in substantially-uniform magnetic field space 131.

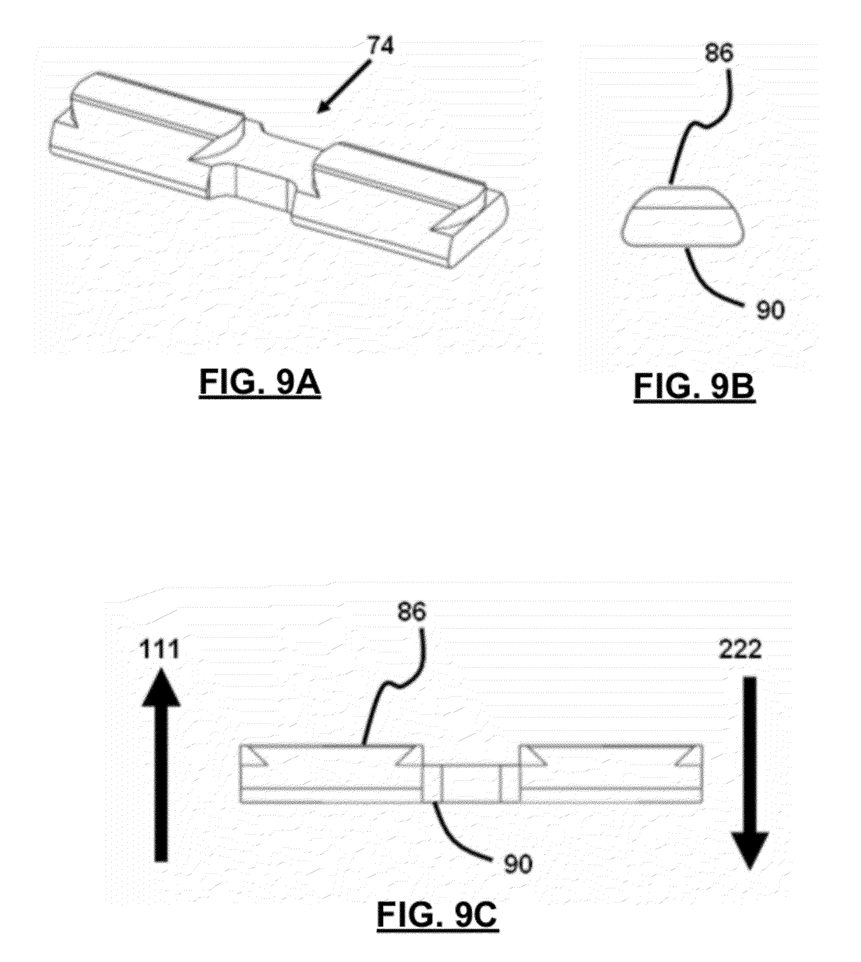

Turning now to FIGS. 9A-9C, a perspective view, an end view, and a side view of one embodiment of a specialized magnet 74 having first end 86 and second end 90 are shown. In this configuration, in first orientation 111, magnet 74 is aligned with substantially-uniform magnetic field space 131 such that a vector originating at second end 90 and terminating at first end 86 is aligned with the direction of magnetic field vectors in substantially-uniform magnetic field space 131. In some embodiments first end 86 may be an N pole and second end 90 may be an S pole. In other embodiments, at first end 86 may be an S pole and second end 90 may be an N pole. In second orientation 222, magnet 74 is aligned with substantially-uniform magnetic field space 131 along its length such that a vector originating at first end 86 and terminating at second end 90 opposes the direction of magnetic field vectors in substantially-uniform magnetic field space 131.

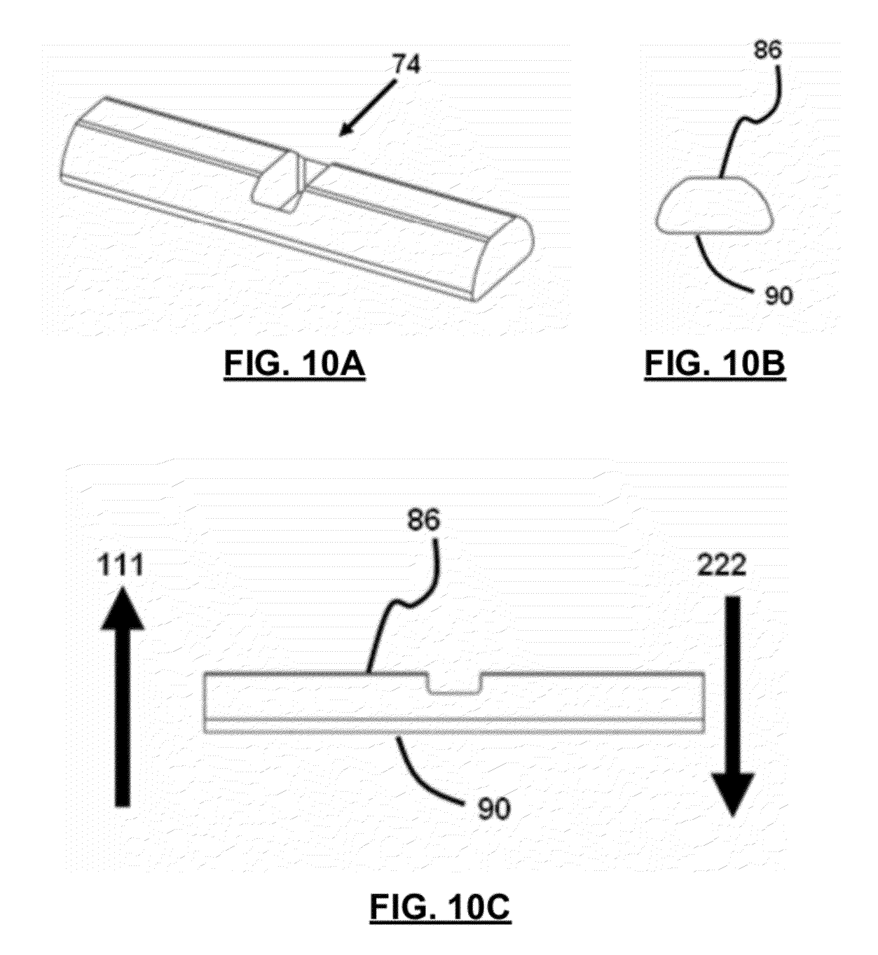

Turning now to FIGS. 10A-10C, a perspective view, an end view, and a side view of another embodiment of a specialized magnet 74 having first end 86 and second end 90 are shown. In this configuration, in first orientation 111, magnet 74 is aligned with substantially-uniform magnetic field space 131 such that a vector originating at second end 90 and terminating at first end 86 is aligned with the direction of magnetic field vectors in substantially-uniform magnetic field space 131. In some embodiments first end 86 may be an N pole and second end 90 may be an S pole. In other embodiments, first end 86 may be an S pole and second end 90 may be an N pole. In second orientation 222, magnet 74 is aligned with substantially-uniform magnetic field space 131 along its length such that a vector originating at first end 86 and terminating at second end 90 opposes the direction of magnetic field vectors in substantially-uniform magnetic field space 131.

Apparatus 34, device 38, magnet 74, or any or all of the three may be sterilized before being used in surgery. In some embodiments, apparatus 34, device 38, or magnet 74 may be placed in a sterile, sealed packaging, which may be removed before surgery. In other embodiments, apparatus 34, device 38, or magnet 74 may be wrapped in a sterile barrier (e.g. a sheet, a paper or a film) before being used in surgery. In embodiments where magnet 74 is not removable from apparatus 34 or device 38, magnet 74 may not be separately sterilized. In other embodiments, magnet 74 may be separately sterilized. In embodiments where magnet 74 is configured to be removable from apparatus 34 or device 38, magnet 74 may be separately sterilized. In embodiments where magnet 74 is configured to be removable from apparatus 34 or device 38 and magnet will not contact the patient during surgery, magnet 74 may not be separately sterilized.

In some embodiments, apparatus 34, device 38, or magnet 74 may be sterilized before being charged. In some embodiments, apparatus 34, device 38, or magnet 74 may be charged first, then sterilized. In other embodiments, apparatus 34, device 38, or magnet 74 may be sterilized when they are charged, but may be unsterilized when they are discharged. In other embodiments, apparatus 34, device 38, or magnet 74 may be sterilized before being discharged.

The various embodiments of the present systems, apparatuses, devices, and methods described in this disclosure can be employed and/or applied for any suitable medical or surgical procedures, including, for example, natural orifice transluminal endoscopic surgery (NOTES), single-incision laparoscopic surgery (SILS), single-port laparoscopy (SLP), and others.

The various illustrative embodiments of systems, apparatuses, devices, and methods described herein are not intended to be limited to the particular forms disclosed. Rather, they include all modifications, equivalents, and alternatives falling within the scope of the claims. For example, though magnet 74 is depicted as a cylinder, a prism, or as having a specialized shape, it is to be understood that magnet 74 may have other shapes. For example, magnet 74 may be conical, pyramidal, annular, or in the shape of a frustum, or any other suitable shape.

The claims are not intended to include, and should not be interpreted to include, means-plus- or step-plus-function limitations, unless such a limitation is explicitly recited in a given claim using the phrase(s) “means for” or “step for,” respectively.

1. A method for magnetically charging a member comprising:
generating a first substantially-uniform magnetic field space with a fixture that is coupled to a member so as to apply a first substantially-uniform magnetic field space to the member, thereby inducing a first magnetic charge in the member, the first magnetic charge having a strength;
coupling the member to a structure configured for use in a medical or surgical procedure; and
inducing a second magnetic charge in the member to diminish the strength of the first magnetic charge.

2. The method of claim 1, further comprising:
sterilizing the structure.

3. The method of claim 1, further comprising:
sterilizing the member.

4. The method of claim 1, further comprising:
sterilizing the structure and the member.

5. The method of claim 1, where the inducing comprises:
decoupling the member from the structure;
generating a second substantially-uniform magnetic field space; and
inducing a second magnetic charge in the member that is diametrical to the first magnetic charge.

6.-10. (canceled)

11. The method of claim 1, where the member is positioned in a magnetizer during the generating and during the demagnetizing.

12.-22. (canceled)

23. A system for magnetically charging a member comprising:
an apparatus comprising a member, where the apparatus is configured to be placed outside the body cavity of a patient and magnetically coupled to a device in the body cavity of a patient through a tissue; and
a fixture configured to generate a substantially-uniform magnetic field space that can induce a magnetic charge in the member.

24. The system of claim 23, where the volume of the apparatus is less than about 64 cubic inches.

25.-28. (canceled)

29. The system of claim 23, where the fixture is configured to generate a first substantially-uniform magnetic field space that can induce a first magnetic charge in the member at a first orientation and that is configured to generate a second substantially-uniform magnetic field space that can induce a second magnetic charge in the member at a second orientation, the second magnetic charge being diametrically opposed to the first magnetic charge.

30. The system of claim 23, where the member is removable from the apparatus.

31. The system of claim 23, where the fixture comprises an electrically conductive coil.

32.-36. (canceled)

37. The system of claim 23, further comprising a capacitive-discharge magnetizer couplable to the fixture.

38. A system for magnetically charging a member comprising:
a sterilized apparatus configured to be placed outside of a body cavity of a patient and magnetically coupled to a sterilized device in the body cavity of a patient through a tissue, where the apparatus comprises a member;
a fixture comprising an electrically-conductive coil, where the coil is configured to generate a substantially-uniform magnetic field space; and
a magnetizer configured to be electrically coupled to the fixture;
where the magnetizer is configured to deliver an electrical current to the coil and the substantially-uniform magnetic field space is configured to induce a magnetic charge in the member.

39. The system of claim 38, where the volume of the sterilized apparatus is less than about 64 cubic inches.

40.-43. (canceled)

44. The system of claim 38, where the magnetizer is a capacitive discharge magnetizer.

45. The system of claim 44, where the magnetizer is configured to deliver at least a 5,000 A electrical current to the coil.

46.-60. (canceled)

61. A system for magnetically charging a member comprising:
a sterilized device comprising a member, where the device is configured to be placed within a body cavity of a patient and magnetically coupled to a sterilized apparatus outside the body cavity of a patient through a tissue;
a fixture comprising at least an electrically-conductive coil, where the coil is configured to generate a substantially-uniform magnetic field space; and
a magnetizer configured to be electrically coupled to the fixture;
where the magnetizer is configured to deliver an electrical current to the coil and the magnetic field space is configured to induce a magnetic charge in the member.

62. The system of claim 61, where the volume of the sterilized apparatus is less than about 64 cubic inches.

63.-65. (canceled)

66. The system of claim 61, where the volume of the sterilized apparatus is less than about 16 cubic inches.

67. The system of claim 61, where the magnetizer is a capacitive discharge magnetizer.

68.-82. (canceled)

83. The system of claim 66, where the member has a first orientation and a second orientation, and where the magnetic charge induced in the member at the first orientation is diametrically opposed to the magnetic charge induced in the member at the second orientation.

84. The system of claim 83, where the member is removable from the sterilized device.

85. The system of claim 84, where the member is sterilized.
